(19) United States
(12) Patent Application Publication
(10) Pub. No.: US 2010/0209963 A1
(43) Pub. Date: Aug. 19, 2010

(54) PHOTO-ELECTRIC DEVICE AND METHOD FOR HIGH THROUGHPUT ACTIVATION, GUIDANCE AND PORATION OF TARGETED CELLS WITH HIGH SPATIAL RESOLUTION

(75) Inventors: Khyati P. Dave, Irvine, CA (US); Samarenda K. Mohanty, Irvine, CA (US); Michael W. Berns, Bonsall, CA (US)

Correspondence Address: Law Offices of Daniel L. Dawes;Dawes Patent Law Group, 5200 Warner Blvd, Ste. 106, Huntington Beach, CA, 92649, US

(73) Assignee: THE REGENTS OF THE UNIVERSITY OF CALIFORNIA, Oakland, CA (US)

(21) Appl. No.: 12/705,517

(22) Filed: Feb. 12, 2010

Related U.S. Application Data
(60) Provisional application No. 61/152,042, filed on Feb. 12, 2009.

(51) Int. Cl. C12Q 1/02; C12N 13/00; C12N 15/00; C12M 1/00; C12N 5/07

(52) U.S. Cl. 435/29; 435/173.1; 435/440; 435/173.7; 435/283.1; 435/287.1; 435/306.1; 435/325

(57) ABSTRACT

The method includes the steps of generating a spatially and/or temporally localized electric field generated on the photoconductive surface, and selectively activating, guiding or porating targeted (excitable) cells at high throughput with high spatial resolution, applied for example to neurons, cardiac and muscle cells. The spatially and/or temporally localized electric field can be established using spatially and/or temporally patterning light with a diffractive element to generate the spatially localized electric field on the photoconductive surface which is sandwiched between two conductive surfaces and applying a selected voltage difference between the two conductive surfaces. The intensity of the light beam can be varied for different processes of activation, guidance or poration without causing cellular damage.

### RELATED APPLICATIONS

The present application is related to U.S. Provisional Patent Application, Ser. No. 61/152,042, filed on Feb. 12, 2009, which is incorporated herein by reference and to which priority is claimed pursuant to 35 USC 119.

### BACKGROUND OF THE INVENTION

1. Field of the Invention

The invention relates to the field of methods and apparatus for providing spatially and/or temporally modulated regions for cellular activation, guidance or poration.

2. Description of the Prior Art

Most of the studies reported in past have been based on exciting (neuronal) cells electrically or optically. For electrical excitation either a single electrode or array of electrodes are used. Light has also been used for genetically targeted activation of neurons if they are transfected with photo-sensitive ion channels or in presence of caged photo-activable compounds. Focused light beams as well as electric fields have also been used independently to enhance and guide neurons and their growth processes. Further, intense focused light beams as well as electric field have been used for poration of cells. While light beam can be spatially configured to guide or transfect several cells (e.g. neurons) in parallel, the high laser power requirements and low throughput has been a hindrance to its applicability. Use of multiple electrodes for excitation, guidance and poration of excitable cells such as neurons is limited due to lack of the ability to reconfigure the electrodes in real time and also due to the complicated fabrication process.

### BRIEF SUMMARY OF THE INVENTION

The illustrated embodiments of the invention include a method comprising the steps of generating a spatially and/or temporally localized electric field generated on the photoconductive surface, and selectively activating, guiding or porating targeted (excitable) cells at high throughput with high spatial resolution. The surface as described below may be those in a culture chamber having a parallel plate capacitor, a capacitor of any kind of shape or construction or a geometric configuration of the surface or surfaces in any three dimensional form desired. For example, the surface may be a planar, cylindrical or spiral surface of any type as long as the modulating light beam can be appropriately directed to the photoconductive surface.

The step of selectively activating and guiding targeted (excitable) cells is applied to neurons, cardiac and muscle cells.

The step of generating the spatially and/or temporally localized electric field comprises the step of spatially and/or temporally patterning light with a diffractive element to generate the spatially localized electric field on the photoconductive surface which is sandwiched between two conductive surfaces and applying a selected voltage difference between the two conductive surfaces.

The step of generating the spatially and/or temporally localized electric field comprises the step of forming a spatially and/or temporally localized photoconductive surface pattern on at least one surface in a capacitor by a spatially and/or temporally modulated light beam directed to the at least one surface to provide cellular activation, guidance or poration regions with selective spatial and/or temporal resolution.

The method further comprises the step of varying the intensity of the light beam for different processes of activation, guidance or poration without causing cellular damage.

The step of generating and selectively activating, guiding or porating targeted (excitable) cells are performed in: 1) controlled modulation of physiological functions of excitable cells in skeletal, cardiac or neuronal systems by opto-electrical excitation (activation) of selected cells with high spatial and temporal accuracy; 2) high-throughput screening for drugs that modulate cellular responses to activation induced depolarization; 3) enhancement and guidance of neuronal growth cones; 4) poration/transfection or 5) lysis of selected cells with high spatial resolution.

The illustrated embodiments of the invention are also to be expressly understood as including an apparatus comprising at least one photoconductive surface; at least one cell disposed on the at least one photoconductive surface, a selectively controllable source of voltage coupled to the at least one surface to generate an electric field at the photoconductive surface, and a selectively controllable light source generating a patterned beam of light onto the at least one photoconductive surface to establish spatially and/or temporally modulated or controlled electric fields in which the at least one cell is disposed to activate, guide or porate the targeted (excitable) cell.

In the illustrated embodiment the apparatus further comprises a multiplicity of cells disposed on the at least one surface for providing selective activation, guiding or poration of a multiplicity of targeted (excitable) cells at high throughput with high spatial resolution.

The multiplicity of cells include neurons, cardiac or muscle cells so that selective activation, guiding or poration is applied to neurons, cardiac or muscle cells.

The apparatus further comprises a diffractive element to generate the spatially and/or temporally localized electric field using a spatially and/or temporally patterned light.

The spatially and/or temporally localized electric field is derived from a spatially and/or temporally localized photoconductive surface pattern on at least one surface in a capacitor by the spatially and/or temporally modulated light beam from the light source directed to the at least one surface to provide cellular activation, guidance or poration regions with selective spatial and/or temporal resolution.

The apparatus further comprises a controller for varying the intensity of the light beam for different processes of activation, guidance or poration without causing cellular damage.

The apparatus is used in combination with a system for: 1) controlled modulation of physiological functions of excitable cells in skeletal, cardiac or neuronal systems by opto-electrical excitation (activation) of selected cells with high spatial and temporal accuracy; 2) high-throughput screening for drugs that modulate cellular responses to activation induced depolarization; 3) enhancement and guidance of neuronal growth cones; 4) poration/transfection or 5) lysis of selected cells with high spatial resolution.

It is be expressly understood the above embodiments can be combined with each other in any one of the logically possible combinations or permutations of the embodiments with each other conceivable.

It is to be further understood that the illustrated embodiments include the cells themselves which are treated by any one of the above embodiments of the method or apparatus.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a sketch of a parallel plate system in which the invention is practiced.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

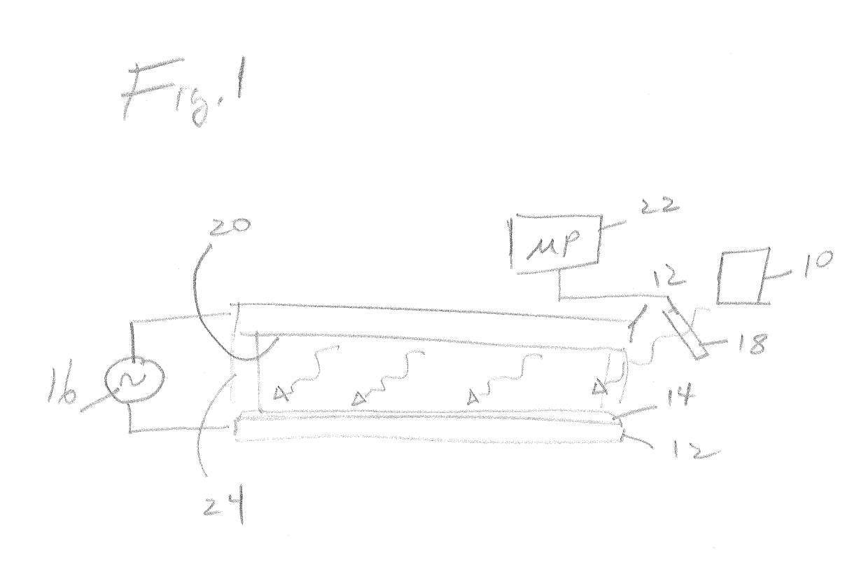

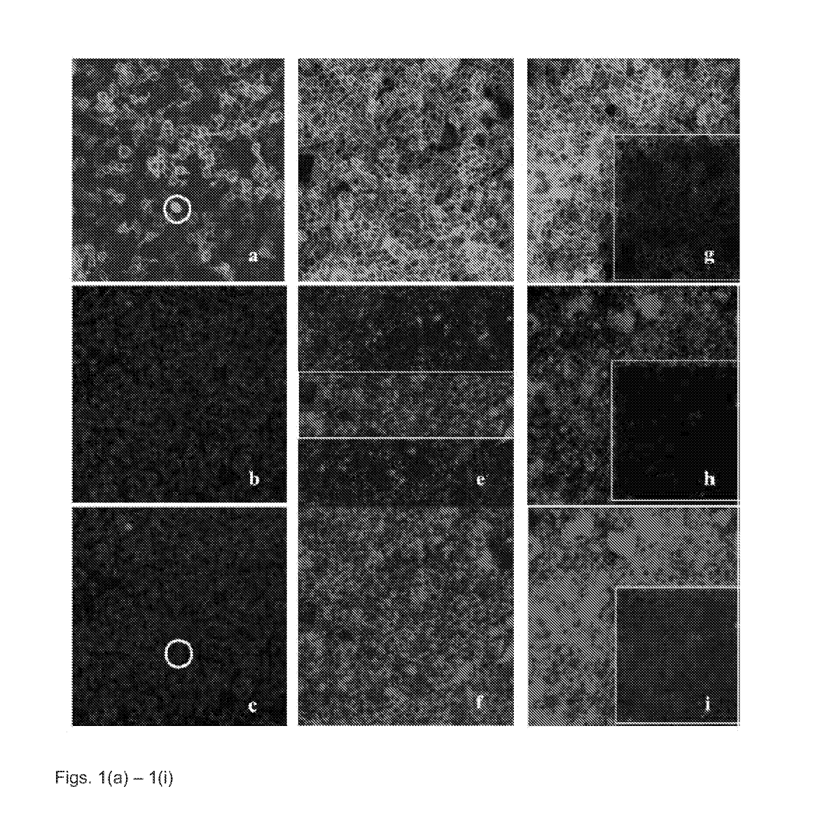

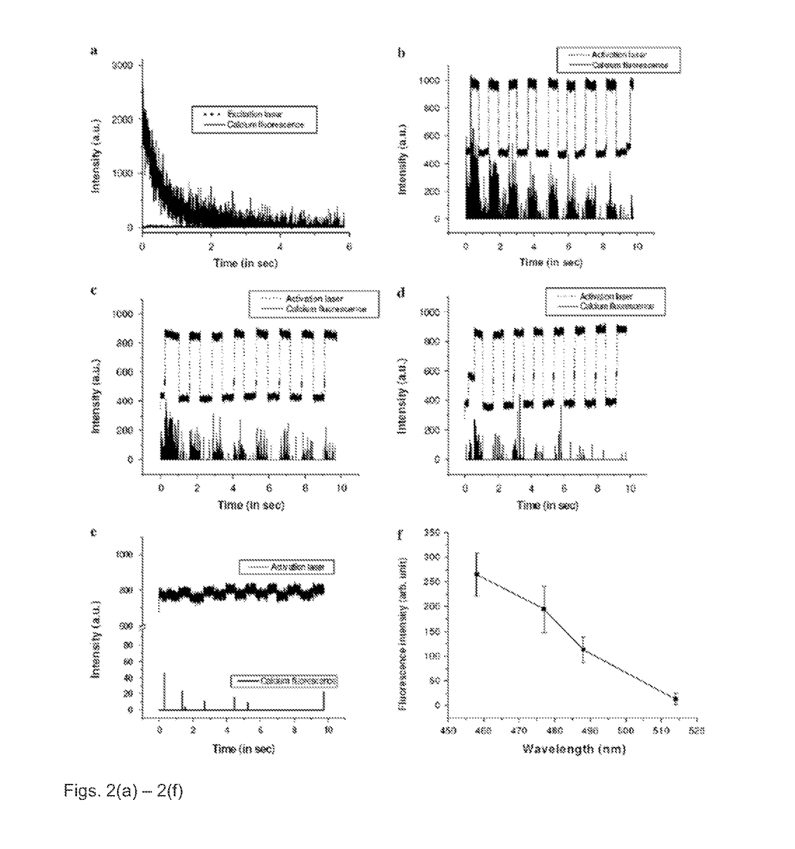

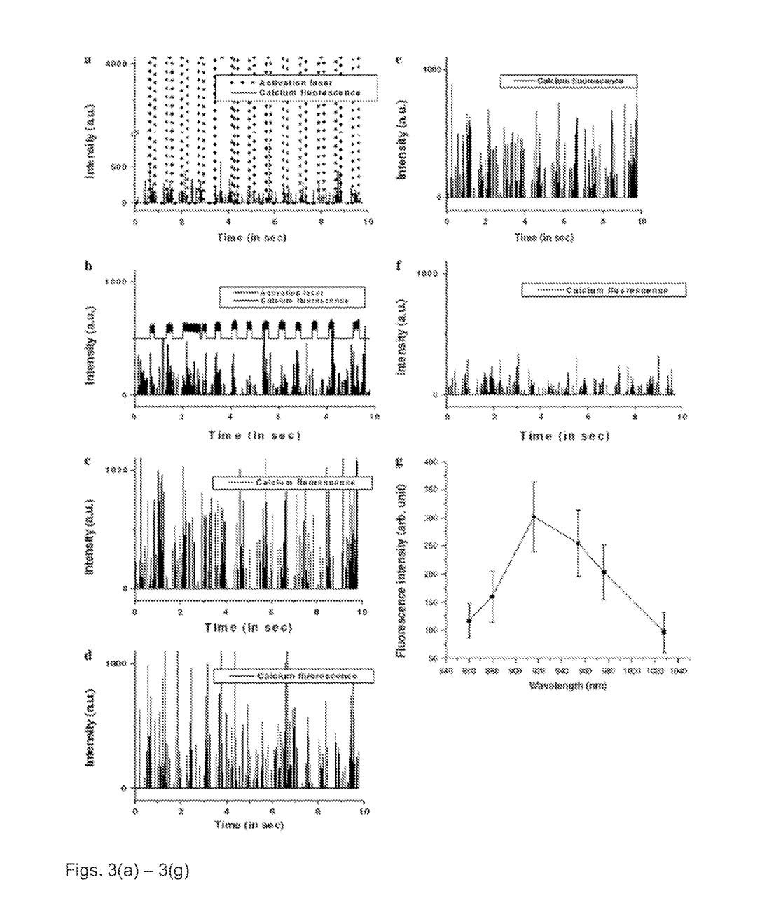

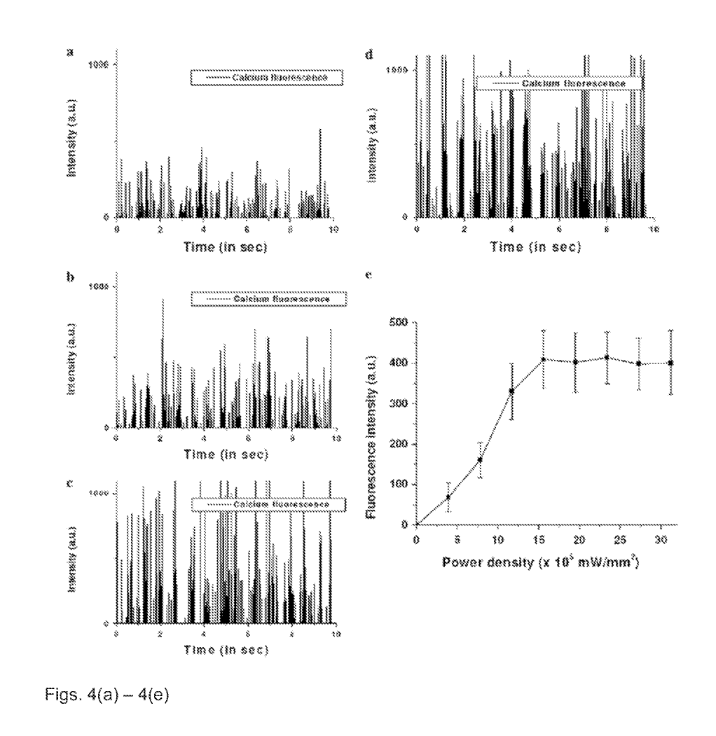

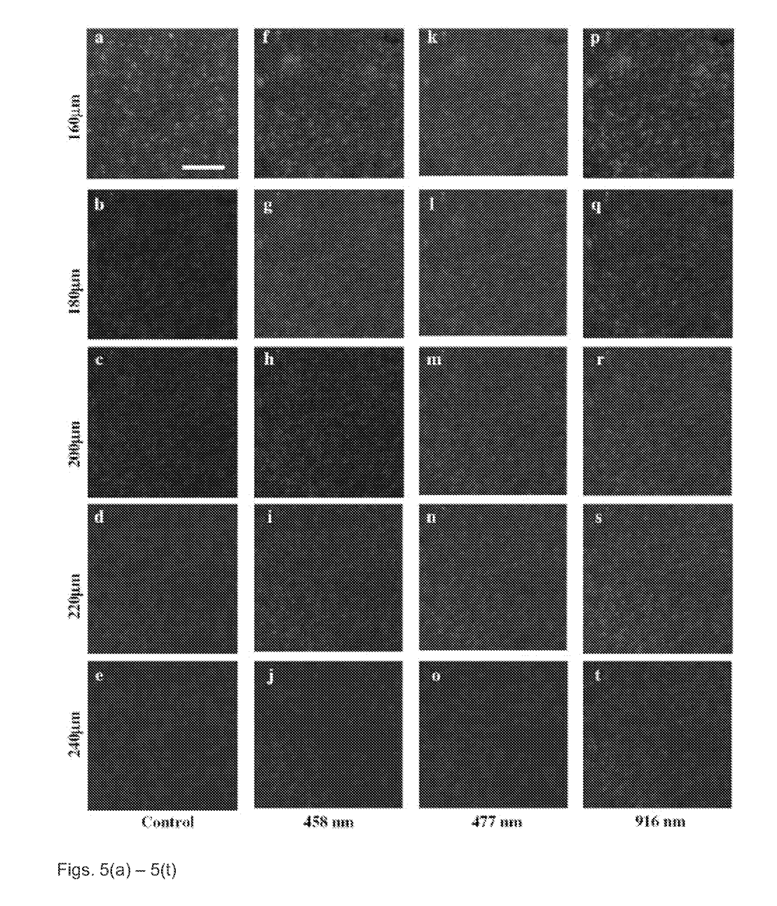

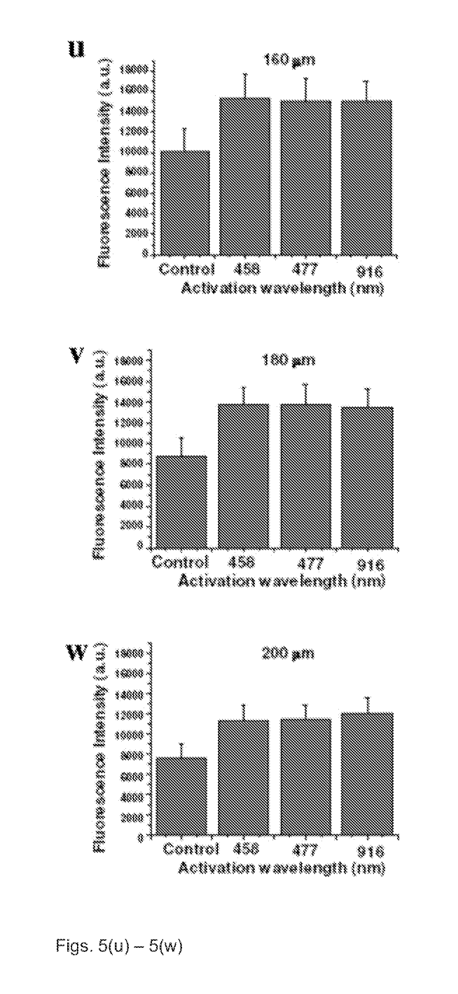

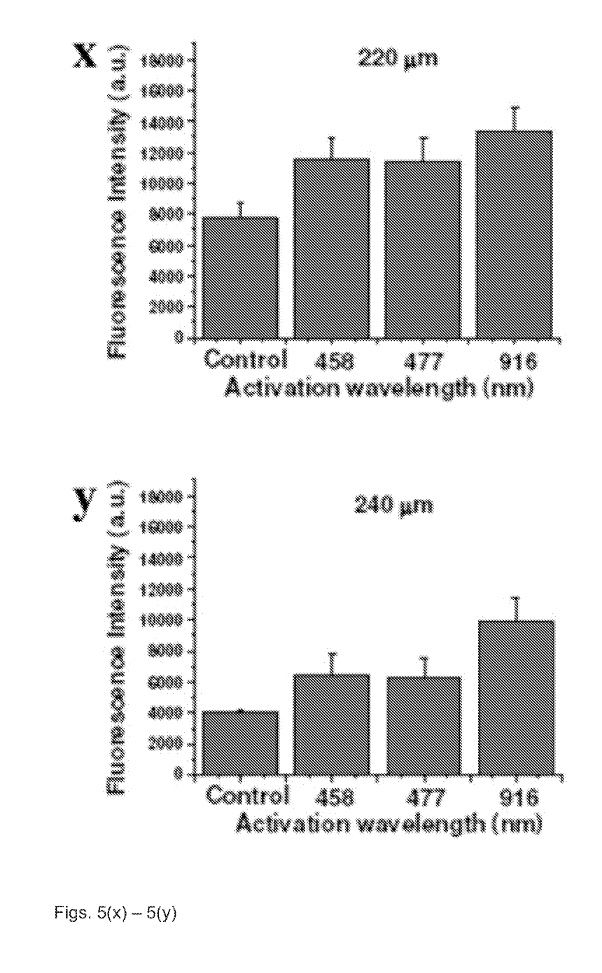

The prior art has made use of either highly intense light beam or electrode(s). The illustrated embodiment of the present invention uses the spatially and/or a temporally localized electric field generated on a photoconductive surface 20 in FIG. 1 by a spatially modulated light beam for selected activation and guidance of targeted (excitable) cells 14 such as neurons with high spatial resolution, and also for poration of biological cells 14, thus making the process simple and also enhancing the throughput. The use of this method will offer an easy-to-use and selective targeting of cells 14 for their excitation, guidance and poration with high spatial and temporal resolution.

The illustrated embodiment of the invention includes a device and method to use spatially modulated light induced electric field for activation, guidance and transfection with high spatial resolution of targeted cells leading to better efficacy for high throughput applications. In order to demonstrate this method, low power laser beam (visible/near IR) from a light source or laser 10 shown in FIG. 1 was spatially modulated by diffractive elements 18 and made to fall on a photoconductive surface 20, e.g. amorphous silicon coated over indium tin oxide (ITO)-glass plate 12 over which the cells 14 are pre-grown. For dynamic change of the light pattern at the sample plane, a spatial light modulator (SLM) 18 was used as a diffractive element, which could be programmed via a computer 22. The sample chamber was made with another ITO-glass plate 12 at the top and an insulating spacer 24 in between the two plates 12. AC (frequency is adjusted by a frequency generator 16) or DC voltage was applied between the two ITO-glass plates 12. As a result, an electric field is induced, on the photoconductive surface 20 at those spatial locations where the light pattern is present. The duration and strength of the electric field is determined by the duration and intensity of the light beam and thus can be controlled with very high temporal resolution.

One of the purposes of the illustrated embodiment of this invention is to use the spatially (and/or temporally) localized electric field generated on the photoconductive surface 20 for selected and high throughput activation and guidance with high spatial resolution of targeted (excitable) cells 14 such as neurons, cardiac and muscle cells, and also for poration of biological cells 14.

The fundamental principle is that light patterned by a diffractive element 18 can generate spatially localized electric field on a photoconductive surface 20 sandwiched between two conductive (e.g. ITO) plates 12 kept at a certain voltage difference. And since electric field is known to excite and guide excitable cells and to porate cells 14, the spatially light mediated localized electric field can be used for selective excitation and or guidance of excitable (e.g. neuronal) cells as well as for transfection of different cells 14.

Since the present invention uses the spatially (and or temporally) localized electric field generated on the photoconductive surface 20 by spatially modulated light beam, the activation, guidance and poration regions can be selected with high spatial resolution. Varying the light power, the different processes of activation, guidance and poration can be realized without causing damage to the cells 14. Further, a low cost laser 10 having power of few mW can generate sufficient electric field to accomplish the above processes in multiple locations thus enhancing the throughput. This method will offer an easy-to-use and selective targeting of cells 14 for their excitation, guidance and poration with high spatial and temporal resolution.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. A method comprising:
generating a spatially and/or temporally localized electric field generated on the photoconductive surface; and
selectively activating, guiding or porating targeted (excitable) cells at high throughput with high spatial resolution.

2. The method of claim 1 where selectively activating and guiding targeted (excitable) cells is applied to neurons, cardiac and muscle cells.

3. The method of claim 1 where generating the spatially and/or temporally localized electric field comprises spatially and/or temporally patterning light with a diffractive element to generate the spatially localized electric field on the photoconductive surface which is sandwiched between two conductive surfaces and applying a selected voltage difference between the two conductive surfaces.

3. The method of claim 1 where generating the spatially and/or temporally localized electric field comprises forming a spatially and/or temporally localized photoconductive surface pattern on at least one surface in a capacitor by a spatially and/or temporally modulated light beam directed to the at least one surface to provide cellular activation, guidance or poration regions with selective spatial and/or temporal resolution.

4. The method of claim 3 further comprises varying the intensity of the light beam for different processes of activation, guidance or poration without causing cellular damage.

5. The method of claim 1 where generating and selectively activating, guiding or porating targeted (excitable) cells are performed in: 1) controlled modulation of physiological functions of excitable cells in skeletal, cardiac or neuronal systems by opto-electrical excitation (activation) of selected cells with high spatial and temporal accuracy; 2) high-throughput screening for drugs that modulate cellular responses to activation induced depolarization; 3) enhancement and guidance of neuronal growth cones; 4) poration/transfection or 5) lysis of selected cells with high spatial resolution.

6. An apparatus comprising:
at least one photoconductive surface;
at least one cell disposed on the at least one photoconductive surface;
a selectively controllable source of voltage coupled to the at least one surface to generate an electric field at the photoconductive surface; and
a selectively controllable light source generating a patterned beam of light onto the at least one photoconductive surface to establish spatially and/or temporally modulated or controlled electric fields in which the at least one cell is disposed to activate, guide or porate the targeted (excitable) cell.

7. The apparatus of claim 6 further comprising a multiplicity of cells disposed on the at least one surface for providing selective activation, guiding or poration of a multiplicity of targeted (excitable) cells at high throughput with high spatial resolution.

8. The apparatus of claim 7 where the multiplicity of cells include neurons, cardiac or muscle cells so that selective activation, guiding or poration is applied to neurons, cardiac or muscle cells.

9. The apparatus of claim 6 further comprising a diffractive element to generate the spatially and/or temporally localized electric field using a spatially and/or temporally patterned light.

10. The apparatus of claim 6 where the spatially and/or temporally localized electric field is derived from a spatially and/or temporally localized photoconductive surface pattern on at least one surface in a capacitor by the spatially and/or temporally modulated light beam from the light source directed to the at least one surface to provide cellular activation, guidance or poration regions with selective spatial and/or temporal resolution.

11. The apparatus of claim 10 further comprises a controller for varying the intensity of the light beam for different processes of activation, guidance or poration without causing cellular damage.

12. The apparatus of claim 6 where the apparatus is used in combination with a system for: 1) controlled modulation of physiological functions of excitable cells in skeletal, cardiac or neuronal systems by opto-electrical excitation (activation) of selected cells with high spatial and temporal accuracy; 2) high-throughput screening for drugs that modulate cellular responses to activation induced depolarization; 3) enhancement and guidance of neuronal growth cones; 4) poration/transfection or 5) lysis of selected cells with high spatial resolution.

13. Cells treated by the method of claim 1.
